(19) United States
(12) Patent Application Publication
(10) Pub. No.: US 2023/0181498 A1
(43) Pub. Date: Jun. 15, 2023

(54) NEW USE OF N,N-BIS-2-MERCAPTOETHYL ISOPHTHALAMIDE

(71) Applicant: EMERAMED LIMITED, Dublin (IE)

(72) Inventor: Ragnar Axel Theodor KLINGBERG, Stockholm (SE)

(21) Appl. No.: 17/917,367

(22) PCT Filed: Apr. 6, 2021
(86) PCT No.: PCT/GB2021/050837

§ 371 Date: Oct. 6, 2022

(30) Foreign Application Priority Data
Apr. 6, 2020 (GB) 2005057.1

(51) Int. Cl. A61K 31/166; A61P 31/12

(52) U.S. Cl. CPC A61K 31/166; A61P 31/12

(57) ABSTRACT

There is provided the compound N,N-bis-2-mercaptoethyl isophthalamide (NBMI), or a pharmaceutically-acceptable salt and/or derivative thereof, for treatment or prevention of viral infections, such as respiratory infections. Such infections may be caused by respiratory viruses, such as rhinoviruses, influenza viruses and coronaviruses, amongst others. NBMI may alleviate symptoms of diseases caused by those viruses, such as cough, dyspnea, respiratory distress and/or respiratory failure, and/or may prevent (e.g. respiratory) virus-induced morbidity and/or mortality.

### FIELD OF THE INVENTION

The present invention relates to new medical uses of the compound N,N-bis-2-mercaptoethyl isophthalamide (NBMI) and pharmaceutically acceptable salts and/or derivatives thereof. In particular, the invention relates to the use of such compounds in treatment or prevention of viral infections, such as respiratory tract infections.

### BACKGROUND OF THE INVENTION

The listing or discussion of an apparently prior-published document in this specification should not necessarily be taken as an acknowledgement that the document is part of the state of the art or is common general knowledge.

A virus is a submicroscopic infectious agent comprising genetic material (DNA or RNA) that is capable of infecting a biological organism. A virus invades and attaches itself to a living cell, after which it multiplies to produce more virus particles (virions), which attach to and enter susceptible cells.

A virus may either kill a cell or alter its functions, the by-products of which processes lead to the infection of other cells. This will then generally lead to what is termed as viral diseases (or a viral infection).

In general, viruses only infect one type of cell, but can be transmitted in various ways, including contact with infected individuals or their bodily secretions, animals (such as arthropods), or inanimate objects. Viruses can also be transmitted by inhalation or swallowing.

Following viral infection, an organism's immune defence system is triggered. Lymphocytes and monocytes attempt to attack and destroy the invasive virus. This is referred to as the body's innate or natural immunity. The innate immune response can often lead a patient feeling unwell or fatigued. If a patient's immune system is compromised, or not effective enough to prevent the spread of a virus, this can lead to severe illness and, in some instances, morbidity and/or death.

Antiviral medicines typically act by interfering with viral replication, for example, by slowing replication rate to increase the likelihood of the innate immune response intervening. There is a clear clinical need for medicines that exhibit better and/or different antiviral properties.

There are many different types of viruses, some of which can seriously affect lung function and cause respiratory illnesses, such as upper respiratory tract infections (URTIs) and lower respiratory tract infections (LRTIs). Viral upper respiratory tract infections, which are the most common acute illnesses caused by respiratory viruses, range from the common cold, to life-threatening illnesses such as epiglottitis. Viral lower respiratory tract infections include conditions that affect the airways, such as bronchitis, or the air sacs (alveoli) at the end of the airways, such as pneumonia.

Common respiratory viruses include corona virus (usually contracted in general usage to ‘coronavirus’, as used hereinafter), influenza virus, respiratory syncytial virus, parainfluenza virus, adenovirus, rhinovirus, human metapneumovirus and enterovirus.

Coronaviruses are a group of related viruses that cause diseases in mammals and birds and inflict upper and lower respiratory tract infections. There are 7 presently-known strains of human coronaviruses:
severe acute respiratory syndrome coronavirus (SARS-CoV),
severe acute respiratory syndrome coronavirus 2 (SARS-CoV-2, previously known as ‘2019-nCoV’ or ‘novel coronavirus 2019’), which the virus that causes coronavirus disease 2019 (COVID-19),
human coronavirus 229E (HCoV-229E), human coronavirus OC43 (HCoV-OC43),
human coronavirus NL63 (HCoV-NL63 or New Haven coronavirus), human coronavirus HKU1,
Middle East respiratory syndrome-related coronavirus (MERS-CoV), previously known as novel coronavirus 2012, and
HCoV-EMC.

In humans, coronaviruses cause respiratory tract infections that are typically mild, but some forms such as SARS-CoV and SARS-CoV-2 can be lethal, which is thought to be due to uncontrolled aggressive deleterious pulmonary inflammation and cellular apoptosis (Fu et al, Virology Sinica, https://doi:10.1007/s12250-020-00207-4 (2020)).

Alveolar epithelial cells are an important target for coronavirus infection in the lungs (Miura and Holmes, J. Luekoc. Biol., 86, 1145 (2009)).

N,N-bis-2-mercaptoethyl isophthalamide (NBMI) was first disclosed in a patent application granted as US patent number U.S. Pat. No. 6,586,600 B2. Its use as a dietary supplement is disclosed in US patent application 2010/0227812, and it is also known to be a chelator of heavy metals, such as mercury, cadmium and lead. Analogues of NBMI have been disclosed in, inter alia, granted U.S. Pat. No. 8,426,368 B2, and international patent applications WO 2011/038385 and WO 2012/121798. Uses of NBMI in the therapeutic treatment of COPD and neurodegenerative disorders, and in the prevention of paracetamol toxicity and/or acute liver failure are disclosed in international patent applications WO 2015/150793, WO 2015/181567 and WO 2018/025049, respectively.

### DESCRIPTION OF THE INVENTION

We have now surprisingly found that NBMI and pharmaceutically-acceptable salts and/or derivatives thereof appear to possess properties relevant for the prevention or treatment of viral infections. These properties may allow for the treatment of a viral disease per se, that is treatment of a viral disease, or a viral infection, possibly by interfering with the replication of the virus within a host, as well as for the treatment of any symptoms of any viral disease or infection. Such properties may also allow for the prevention of the onset of such an infection or disease, the protection of cells in a host from (e.g. further) viral disease, prevention or arrest of the spread of viral disease or infection (within a single host, or from one host to a new host), or for the prevention of reactivation of a virus after latency in a host.

According to a first aspect of the invention there is provided NBMI, or a pharmaceutically-acceptable salt and/or derivative thereof, for use in a method of treatment or prevention of a viral infection.

In an alternative first aspect of the invention there is provided the use of the compound NBMI, or a pharmaceutically-acceptable salt and/or derivative thereof, in the manufacture of a medicament for the treatment of a viral infection.

In a further alternative first aspect of the invention there is provided a method of treatment of a viral infection, which method comprises the administration of an effective amount of NBMI, or a pharmaceutically-acceptable salt and/or derivative thereof, to a patient in need thereof (i.e. a patient in need of such treatment or prevention).

Accordingly, the administration of NBMI, or pharmaceutically-acceptable salts and/or derivatives thereof, may be effective in treatment or prevention of diseases or infections caused by viruses, such as adenoviridae (e.g. adenovirus), papillomaviridae (e.g. human papillomavirus), polyomaviridae (e.g. BK virus; JC virus), herpesviridae (e.g. herpes simplex, type 1; herpes simplex, type 2; varicella-zoster virus; Epstein— Barr virus; human cytomegalovirus; human herpes virus, type 8), poxviridae (e.g. smallpox), hepadnaviridae (e.g. hepatitis B virus), parvoviridae (e.g. parvovirus B19), astroviridae (e.g. human astrovirus), caliciviridae (e.g. norovirus; Norwalk virus), picornaviridae (e.g. coxsackievirus, hepatitis A virus; poliovirus; rhinovirus), coronaviridae (e.g. severe acute respiratory syndrome viruses), flaviviridae (e.g. hepatitis C virus; yellow fever virus; dengue virus; West Nile virus; tick-borne encephalitis virus), retroviridae (e.g. human immunodeficiency virus; HIV), togaviridae (e.g. rubella virus), arenaviridae (e.g. Lassa virus), bunyaviridae (e.g. hantavirus; Crimean-Congo hemorrhagic fever virus; Hantaan virus), filoviridae (e.g. Ebola virus; Marburg virus; Ravn virus), orthomyxoviridae (e.g. influenza viruses, including influenza A virus (e.g. H1N1 and H3N2 viruses), influenza B virus or influenza C virus), paramyxoviridae (e.g. measles virus; mumps virus; parainfluenza virus, respiratory syncytial virus), rhabdoviridae (e.g. rabies virus), hepeviridae (e.g. hepatitis E virus), reoviridae (e.g. rotavirus; orbivirus; coltivirus; Banna virus), as well as viruses not assigned to families, such as hepatitis D virus.

It is preferred that the virus causing the infection and/or the disease is a virus of the respiratory tract.

Viral respiratory tract infections include viral upper respiratory infections, including infections in the upper respiratory tract, including nose, sinus, pharynx, or larynx, and viral lower respiratory infections, including infections in the windpipe and lungs. Common symptoms of such infections, including diseases caused by the infection, are cough, sneezing, nasal discharge, nasal congestion, fever, sore throat, headache, facial pressure, fatigue or weakness, myalgias, malaise, dyspnea, respiratory distress, respiratory failure and/or conjunctivitis. In particular embodiments, the symptoms caused by the viral infection and/or disease include one or more of cough, dyspnea, respiratory distress and/or respiratory failure.

In particular embodiments, the viral respiratory tract infection is a viral upper respiratory tract infection.

Accordingly, the administration of NBMI, or pharmaceutically-acceptable salts and/or derivatives thereof, may be effective in treatment or prevention of respiratory diseases or infections caused by respiratory viruses, such as adenoviridae (e.g. adenovirus), picornaviridae (e.g. enterovirus; parechovirus; rhinovirus), coronaviridae (e.g. MERS coronavirus; SARS coronavirus), orthomyxoviridae (e.g. influenza viruses, including influenza A virus (e.g. H1N1 and H3N2 viruses), influenza B virus, influenza C or influenza D virus), paramyxoviridae (e.g. human metapneumovirus; measles virus; mumps virus; parainfluenza virus, respiratory syncytial virus), as well as viruses not assigned to families.

In particular embodiments, the viral respiratory tract infection is caused by a virus selected from the group consisting of an influenza virus, a respiratory syncytial virus, a parainfluenza virus, a coronavirus, an adenovirus, a rhinovirus, or an enterovirus.

Particular respiratory viruses that may be mentioned include viruses associated with the common cold (such as rhinoviruses, adenoviruses, human respiratory syncytial viruses, enteroviruses, human parainfluenza viruses) all influenza viruses mentioned above and, more particularly, coronaviruses, such as those mentioned hereinbefore (including SARS coronaviruses, such as SARS-CoV and, especially, SARS-CoV-2).

In this respect, NBMI or a pharmaceutically-acceptable salt and/or derivative thereof may prevent the onset of respiratory infections or diseases cause by a viruses that are known to cause, or may cause, viral respiratory diseases, including common colds, influenza, sinus infections, tonsillitis, laryngitis, bronchitis, pneumonia and other manifestations of SARS, and bronchiolitis.

Further, NBMI or a pharmaceutically-acceptable salt and/or derivative thereof may treat, and/or arrest the progress of, diseases that are being, or have been, caused by respiratory viruses. NBMI or a pharmaceutically-acceptable salt and/or derivative thereof may also treat and/or prevent the damage that is being, or has been, caused by such viruses, which includes treating and/or preventing the symptoms of such viral respiratory diseases, which include common colds, sinus infections, tonsillitis, laryngitis, bronchitis, pneumonia, and bronchiolitis.

It is believed based on the evidence presented hereinafter that NBMI may prevent and/or treat more severe respiratory viral infections, such as influenza, SARS and, more particularly, diseases such as Covid-19.

In this respect, NBMI and pharmaceutically acceptable salts and/or derivatives thereof may prevent or arrest the progress of (e.g. respiratory) virus-induced morbidity and/or mortality.

Unless indicated otherwise, all technical and scientific terms used herein have the same meaning as commonly understood by one of ordinary skill in the art to which this invention pertains.

Unless specified otherwise, NBMI or pharmaceutically-acceptable salts and/or derivatives thereof may be referred to herein as “compounds of the invention”.

Particular features and embodiments described in relation to a given aspect of the invention should, unless the context indicates otherwise, be regarded as having been disclosed in combination with any and all other particular features and embodiments of that aspect of the invention.

For the avoidance of doubt, the compound NBMI as described herein may also be referred to by the trade name Irminix® or by the international non-proprietary name (INN) Emeramide. The structure of the compound (in non-salt form) is represented below.

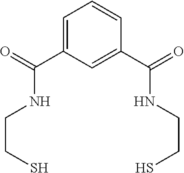 [SMILES: O=C(NCCS)c1cccc(C(=O)NCCS)c1]

Compounds of the invention may be obtained commercially or may be prepared using techniques known to those skilled in the art, such as those described in the disclosures referneced herein. For example, NBMI may be prepared in accordance with the procedure described in U.S. Pat. No. 6,586,600 B2, the contents of which (in particular, the procedures as described in the examples provided therein) are hereby incorported by reference.

Pharmaceutically acceptable salts as referred to within the scope of the present invention include acid addition salts and base addition salts. Such salts may be formed by conventional means; for example, by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. under reduced pressure, by freeze-drying or by filtration). Salts may also be prepared by exchanging a counter-ion of a compound of the invention in the form of a salt with another counter-ion, for example, using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, a-hydroxybutyrate, lactate, tartrate, phenylacetate, mandelate, phenylpropionate, phenylbutyrate, benzoate, chlorobenzoate, methyl benzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalenedisulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic and inorganic bases (such as aluminium hydroxide). More particularly, base addition salts that may be mentioned include Mg, Ca and, most particularly, K and Na salts.

Particular pharmaceutically-acceptable salts that may be mentioned (in particular, when forming salts of NBMI) include base addition salts, such as those formed with alkali metals (e.g. salts formed with Na or K).

The skilled person will understand that references to pharmaceutically-acceptable derivatives of NBMI will include compounds formed by derivatisation of NBMI following procedures known to those skilled in the art, such as through modification of one or more of the —NH— and/or —SH moieties therein. For example, references to such derivatives may include derivatisation of —NH— moieties through alkylation thereof (e.g. to form an —NR¹— moiety, wherein R¹ represents C₁₋₃ alkyl optionally substituted with one or more fluoro group, such as wherein R¹ represents methyl) and/or derivatisation of —SH moieties through alkylation thereof (e.g. to form an —SR² moiety, wherein R² represents C₁₋₃ alkyl optionally substituted with one or more fluoro group, such as wherein R² represents methyl) or esterification thereof (e.g. to form an —SC(O)R³ moiety, wherein R³ represents C₁₋₃ alkyl optionally substituted with one or more fluoro group, such as wherein R³ represents methyl, or another moiety resulting from reaction of a pharmaceutically-acceptable compound capable of forming such thioesters, such as a pharmaceutically-acceptable carboxylic acid or ester).

Particular pharmaceutically-acceptable derivatives of NBMI that may be mentioned include the di-sulphide bridged additions of glutathione, cysteine, alphadihydrolipoic acid, cystamine, thiolphosphate, 5′-thioladenosine, L-homocysteine, co-enzyme A, 2-mercaptoethanol and dithiothreitol. Such derivatives may be prepared by analogy to the procedures described in, for example, US patent application 2011/0237776, the contents of which are hereby incorporated by reference.

For the avoidance of doubt, references to “N,N-bis-2-mercaptoethyl isophthalamide (NBMI), or a pharmaceutically-acceptable salt and/or derivative thereof” will indicate that NBMI may be present in the form of a pharmaceutically-acceptable salt thereof, a pharmaceutically-acceptable derivative thereof, or a pharmaceutically-acceptable salt of a pharmaceutically-acceptable derivative thereof.

Particular compounds of the invention that may be mentioned include NBMI and pharmaceutically-acceptable salts thereof.

More particular compounds of the invention that may be mentioned include NBMI.

For the avoidance of doubt, compounds of the invention may exist as solids, and thus the scope of the invention includes all amorphous, crystalline and part-crystalline forms thereof. Where compounds of the invention exist in crystalline and part-crystalline forms, such forms may include solvates, which are included in the scope of the invention. Compounds of the invention may also exist in solution.

The present invention also embraces isotopically-labelled compounds of the invention, which are identical, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, references to the compounds of the invention also includes deuterated compounds, i.e. in which one or more hydrogen atoms are replaced by the hydrogen isotope deuterium.

The skilled person will understand that references herein to the “treatment” of a particular condition (or, similarly, to “treating” that condition) take their normal meanings in the field of medicine. In particular, the terms may refer to achieving a reduction in the severity of one or more clinical symptom associated with the condition.

The skilled person will understand that references herein to “prevention” of a particular condition (and, similarly, to “preventing” that condition) take their normal meanings in the art. In particular, these terms may refer to achieving a reduction in the likelihood of developing the relevant condition or symptoms associated with the relevant condition (for example, a reduction of at least 10% when compared to the baseline level, such as a reduction of at least 20% or, more particularly, a reduction of at least 30%). Similarly, the term “preventing” may also be referred to as “prophylaxis” of the relevant condition, and vice versa.

As used herein, references to “patients” will refer to a living subject being treated, including mammalian (in particular, human) patients, and as such “patients” may also be referred to as “subjects”, and vice versa. References to “patients” (and therefore also to “subjects”) also should be considered to refer to individuals displaying no symptoms of the relevant condition, for whom compounds of the invention may be used as a preventative or prophylactic measure (as defined herein above).

For the avoidance of doubt, references to “patients” may also include references to animals, such as non-mammalian animals (e.g. birds) and, particularly, mammalian animals (e.g. cats, dogs, rabbits, rodents, horses, sheep, pigs, goats, cows, primates, and the like).

As used herein, the term “effective amount” will refer to an amount of a compound that confers the desired therapeutic effect on the treated subject (i.e. the desired treatment or prevention, as described herein). The effect may be objective (i.e. measurable by some test or marker) or subjective (i.e. the subject gives an indication of and/or feels an effect).

The skilled person will understand that, when employed in the uses and methods described herein, compounds of the invention may be administered in a manner allowing for systemic absorption, which absorption may occur via a number of possible routes; for example, compounds of the invention may be administered orally, intravenously or intraarterially, intramuscularly, cutaneously, subcutaneously, transmucosally (e.g. sublingually or buccally), rectally, transdermally, nasally, pulmonarily (e.g. by inhalation, tracheally or bronchially), or by any other parenteral route, in the form of a pharmaceutical preparation comprising the compound in a pharmaceutically acceptable dosage form. In particular, compounds of the invention may be administered orally, rectally or intravenously (e.g. by intravenous infusion).

The compounds of the invention will generally be administered in the form of one or more pharmaceutical formulations in admixture with a pharmaceutically acceptable excipient, which may be selected with due regard to the intended route of administration and standard pharmaceutical practice. Such pharmaceutically acceptable excipients may be chemically inert to the active compounds and may have no detrimental side effects or toxicity under the conditions of use. Such pharmaceutically acceptable excipients may also impart an immediate (e.g. rapid), or a modified (e.g. delayed), release of the compounds of the invention.

Suitable pharmaceutical formulations may be commercially available or otherwise are described in the literature (see, for example, Remington The Science and Practice of Pharmacy, 19th ed., Mack Printing Company, Easton, Pennsylvania (1995) and Martindale—The Complete Drug Reference (35th Edition), and the documents referred to therein), the relevant disclosures in all of which documents are hereby incorporated by reference. Otherwise, the preparation of suitable formulations may be achieved non-inventively by the skilled person using routine techniques. Suitable pharmaceutical formulations for use with the compounds of the invention are also described in US patent application 2010/0227812.

Accordingly, in a further aspect of the invention, there is provided a pharmaceutical composition comprising N,N-bis-2-mercaptoethyl isophthalamide, or a pharmaceutically acceptable salt and/or derivative thereof, and optionally one or more pharmaceutically-acceptable excipient, for use in:
(a) the treatment or prevention of viral infections, such as respiratory tract infections as described herein; or
(b) the treatment or prevention of symptoms associated with viral infections, such as respiratory tract infections as described herein.

In an alternative aspect of the invention, there is provided a method for:
(a) the treatment or prevention of viral infections, such as respiratory tract infections as described herein; or
(b) the treatment or prevention of symptoms associated with viral infections, such as respiratory tract infections as described herein,
which method comprises the administration of an effective amount of a pharmaceutical composition comprising N,N-bis-2-mercaptoethyl isophthalamide, or a pharmaceutically acceptable salt and/or derivative thereof, and optionally one or more pharmaceutically-acceptable excipient, to a patient in need thereof.

For the avoidance of doubt, unless otherwise stated, all embodiments and particular features described in relation to particular aspects of the invention (and combinations thereof) will also apply to all other aspects of the invention, as appropriate.

The skilled person will understand that references herein to pharmaceutically acceptable excipients include references to pharmaceutically acceptable adjuvants, diluents and/or carriers, which adjuvants, diluents and carriers will be known to those skilled in the art (as described herein).

As described herein, pharmaceutical formulations may be prepared in a manner suitable for the desired route of administration, using techniques and materials known to those skilled in the art. In particular, pharmaceutical formulations may take the form of oral formulations or intravenous formulations (or formulations, e.g. concentrated formulations, suitable for use in the preparation of intravenous formulations).

For example, when intended for oral administration, pharmaceutical formulations comprising compounds of the invention may be provided in the form of a tablet, or an oral powder or solution, each optionally comprising suitable excipients, which may be prepared using techniques known to those skilled in the art. Similarly, when intended for intravenous (I.V.) administration, pharmaceutical formulations comprising compounds of the invention may be provided in the form of solutions suitable for I.V. administration, or as solutions suitable for the preparation of solutions suitable for I.V. administration, which may be prepared using techniques known to those skilled in the art. Similarly, when intended for rectal administration, pharmaceutical formulations comprising compounds of the invention may be provided in the form of a tablet (e.g. a suppository), or a powder or solution, each optionally comprising suitable excipients, which may be prepared using techniques known to those skilled in the art.

Depending on the patient to be treated, the route of administration and the severity of the condition, compounds of the invention may be administered at varying therapeutically effective doses (to the relevant patient in need thereof). Suitable doses may be determined by the skilled person using routine techniques, such as by routine dose titration studies and the like.

Similarly, the amount of the compounds of the invention included in the relevant pharmaceutical formulations may be determined based on the desired dosage of the compound of the invention, the ease of formulation and the route of administration (which may in turn determine the availability of the compound of the invention for systemic absorption).

Suitable doses of the compounds of the invention may include dosages (e.g. for I.V. administration) in the range of from about 0.05 to 300 mg/kg, such as from about 0.5 to about 200 mg/kg (e.g. about 1 to about 100 mg/kg, such as about 5 mg/kg or about 50 mg/kg).

In particular, such doses may be administered by I.V. administration, orally or rectally (e.g. by I.V. administration), over a period of time, such as about 15 minutes, one hour or several hours (e.g. one hour). Moreover, such doses may be repeated as necessary, such as in the form of periodic, sequential infusions, which infusions may be of decreasing dose.

For the avoidance of doubt, wherever the word “about” is employed herein, for example in the context of amounts (e.g. doses of active ingredients), it will be appreciated that such variables are approximate and as such may vary by ±10%, for example ±5% and preferably ±2% (e.g. ±1%) from the numbers specified herein.

For the avoidance of doubt, the skilled person will understand that dose of compounds of the invention administered (e.g. to a human) should be sufficient to effect the desired therapeutic response or preventative effect within (and over) a reasonable timeframe. For example, compounds of the invention may be provided in a form suitable for rapid (i.e. quick or immediate) release of the active ingredient(s), such as in the form of a rapidly disintegrating tablet, which tablets may be formulated using techniques and materials known to those skilled in the art.

In any event, the skilled person will be able to determine routinely the actual dosage which will be most suitable for an individual patient. While the above-mentioned dosages are exemplary of the average case, there can of course be individual instances where higher or lower dosage ranges are merited, and such are within the scope of the invention.

Administration of the compounds of the invention may be continuous (e.g. by continuous I.V. infusion) or intermittent (e.g. by bolus injection or through periodic administration of a tablet or solution, orally or rectally, such as by bolus injection or through periodic administration of a tablet or solution), or may be provided in the form of a single dose (e.g. by injection or through administration of a tablet or solution). The dosage form may also be determined by the timing and frequency of administration, and vice versa.

In particular, in the prevention of viral (e.g. respiratory tract) infections, NBMI may be administered on a continuous (e.g. daily, twice daily, etc.) basis to prevent the onset of symptoms associated with diseases caused thereby.

In the uses and methods described herein, the compounds of the invention may also be combined with one or more active ingredients that are potentially useful, or have been indicated for use, in the treatment of viral infections, such as respiratory tract infections, e.g. upper respiratory tract infections.

In a further aspect of the invention, administration of the compounds of the invention may be combined with administration (e.g. concomitant or sequential administration) of one or more other therapeutic agent that possess antiviral properties useful in the treatment or prevention of viral infections, such as respiratory tract infections, as known to those skilled in the art, such as those described herein below.

As used herein, references to “sequential administration” may refer to separate administration of the therapeutic agents as part of the same medical intervention (e.g. within four hours, such as within two hours or, particularly within one hour, of each other).

Therapeutic agents that may be used in conjunction with NBMI, or a pharmaceutically-acceptable salt and/or derivative thereof, in the treatment of infections that are being, or have been, caused by (e.g. respiratory) viruses in accordance with the invention, include more the variously-applied standard treatments for viral (e.g. respiratory tract) infections, including antiviral medicines (e.g. oseltamivir, zanamivir, amantadine, rimantadine), antiinflammatory agents (e.g. NSAIDs, such as ibuprofen, ketorolac, naproxen and the like), analgesics (e.g. paracetamol or opioids), antitussive agents (e.g. dextromethorphan) and/or vaccination (if available).

Subjects may thus also (and/or may be already) be receiving said other therapeutic agents, by which we mean receiving a prescribed dose of one or more of those other therapeutic agents, prior to, in addition to, and/or following, treatment with NBMI or a pharmaceutically-acceptable salt and/or derivative thereof.

Accordingly, in a further aspect of the invention, there is provided a pharmaceutical formulation comprising:
(a) N,N-bis-2-mercaptoethyl isophthalamide, or a pharmaceutically-acceptable salt and/or derivative thereof; and
(b) one or more other therapeutic agent that is useful in the treatment or prevention of viral infections, such as respiratory tract infections, and optionally one or more pharmaceutically-acceptable excipients, for use in the treatment of e.g. upper respiratory tract infections.

Further, in another aspect of the invention, there is provided a kit-of-parts comprising components:
(A) a pharmaceutical formulation comprising N,N-bis-2-mercaptoethyl isophthalamide, or a pharmaceutically-acceptable salt and/or derivative thereof, and optionally one or more pharmaceutically-acceptable excipients; and
(B) a pharmaceutical formulation comprising one or more other therapeutic agent that is useful in the treatment or prevention of viral infections, such as respiratory tract infections, and optionally one or more pharmaceutically-acceptable excipients, which components (A) and (B) are each provided in a form that is suitable for administration in conjunction with the other, for use in the treatment of e.g. upper respiratory tract infections.

For the avoidance of doubt, all embodiments and particular features described in relation to previously mentioned aspects of the invention (and combinations thereof) will also apply to these aspects of the invention.

The skilled person will understand that the kits-of-parts described herein may comprise more than one formulation including an appropriate quantity/dose of the compounds of the invention, and/or more than one formulation including an appropriate quantity/dose of one or more other therapeutic agent capable of treating or preventing viral infections, such as respiratory tract infections (or symptoms thereof), in order to provide for repeat dosing. If more than one formulation (comprising either active compound) is present, such formulations may be the same, or may be different in terms of the dose of either compound, chemical composition(s) and/or physical form(s).

With respect to the kits of parts as described herein, by “administration in conjunction with” (and similarly “administered in conjunction with”) we include that respective formulations comprising the compounds of the invention, and one or more other therapeutic agent capable of treating or preventing viral infections, such as respiratory tract infections, are administered, sequentially, separately or simultaneously, as part of the same medical intervention.

Therefore, in relation to the present invention, the term “administration in conjunction with” (and similarly “administered in conjunction with”) includes that the two active ingredients (i.e. a compound of the invention, and one or more other therapeutic agent capable of treating or preventing viral (e.g. respiratory tract) infections) are administered (optionally repeatedly) either together, or sufficiently closely in time, to enable a beneficial effect for the patient, that is greater, over the course of the treatment of the relevant condition, than if either a formulation comprising a compound of the invention, or a formulation comprising one or more other therapeutic agent capable of treating or preventing viral infection, such as respiratory tract infections, or pharmaceutically acceptable salt thereof, are administered (optionally repeatedly) alone, in the absence of the other component, over the same course of treatment. Determination of whether a combination provides a greater beneficial effect in respect of, and over the course of, treatment or prevention the relevant condition may be achieved routinely by the skilled person.

Further, in the context of the present invention, the term “in conjunction with” includes that one or other of the two formulations may be administered (optionally repeatedly) prior to, after, and/or at the same time as, administration of the other component.

When used in this context, the terms “administered simultaneously” and “administered at the same time as” include that individual doses of a compound of the invention and one or more other therapeutic agent capable of treating or preventing a viral, such as a respiratory, disease and symptoms thereof are administered within 24 hours (e.g. within 12 hours, 6 hours, 3 hours, 2 hours, 1 hour, 45 minutes, 30 minutes, 20 minutes or 10 minutes) of each other.

Thus, in relation to a further aspect of the invention, there is also provided a kit-of-parts comprising:
(I) one of components (A) or (B) as described in the previous aspect of the invention; and
(II) instructions to use that component in combination with the other of the two components,

for use in the treatment of viral infections, such as respiratory tract infections, e.g. upper respiratory tract infections.

The pharmaceutical formulation or kit-of parts, as appropriate, may be for use in:
(1) the treatment or prevention of viral infections, such as respiratory tract infections, e.g. upper respiratory tract infections, as defined herein; and/or
(2) the treatment or prevention of symptoms associated with viral infections, such as respiratory tract infections, e.g. upper respiratory tract infections, as defined herein, by administering a therapeutically effective amount of NBMI, or a pharmaceutically acceptable salt and/or derivative thereof, to a patient in need thereof (e.g. as a pharmaceutical formulation or kit-of-parts, as appropriate, containing the relevant therapeutically effective amount).

As described herein, pharmacetical formulations (including those containing more than one active ingredient as described herein) may be prepared using techniques known to those skilled in the art. Similarly, a kit-of-parts (as described in herein) may be prepared using techniques known to those skilled in the art.

According to further aspects of the invention, there is provided:
a method of preparing a pharmaceutical formulation comprising components (a) and (b) as described hereinbefore, comprising bringing into admixture (i.e. into the same formulation) components (a) and (b), optionally together with one or more pharmaceutically acceptable excipients; and
a method of preparing a kit-of-parts comprising components (A) and (B) as described hereinbefore, comprising bringing into association components (A) and (B).

For the avoidance of doubt, by bringing the components “into association with” each other, we include that components (A) and (B) of the kit-of-parts as described hereinbefore may be:
provided as separate formulations (i.e. independently of each other), which are subsequently brought together for use in conjunction with each other in combination therapy; or
packaged and presented as separate components of a “combination pack” for use in conjunction with each other in combination therapy.

When employed in the uses and methods described herein, compounds of the invention may have the advantage that, in the treatment or prevention of viral (e.g. respiratory) diseases or viral (e.g. respiratory tract) infections associated with viral (e.g. respiratory) diseases, may be more convenient for the physician and/or patient than, be more efficacious than, be less toxic than, have a broader range of activity than, be more potent than, produce fewer side effects than, or that it may have other useful pharmacological properties over, similar treatments or preventative measures known in the prior art.

### EXAMPLES

The invention is further illustrated, but not limited, by the following examples.

### Example 1

### Treatment of Patient I

A man residing in the USA reported taking doses of up to 300 mg/day NBMI almost daily since 2006. The man, who will be turning 80 years old in September 2020, has now been taking NBMI almost daily for approximately 14 years, during which time he has not become unwell with common cold-like and/or flu-like illnesses even though his wife, who worked as a registered nurse was inflicted with them on many occasions.

Before taking NBMI, the man regularly became sick with such illnesses due to his work as a university professor and being an avid outdoorsman.

### Example 2

### Treatment of Patient II

A woman residing in the USA, the wife of Patient I, reported that she was often inflicted with common cold-like and/or flu-like symptoms due to her profession as a registered nurse. In 2010, the woman started to take NBMI daily and has shown great decrease in having flu-like symptoms.

The woman now takes NBMI daily and has not experienced any such symptoms over the past 5 years.

### Example 3

### Treatment of Patient with ME/CFS I

A man residing in Sweden and diagnosed with myalgic encephalomyelitis/chronic fatigue syndrome (ME/CFS), reported that he started to use NBMI extensively in 2010 starting with 600 mg/day for 1.5 months, then 400 mg/day for 1.5 months and then 300 mg/day for three months.

Due to ME/CFS, the man was often inflicted with viral infections. Immediately after starting the treatment with NBMI, the man noticed that the viral infections became fewer and that their intensity was less severe (e.g. less pain, headache, fatigue, time bedridden) and that the recovery time was quicker.

After halting the treatment in summer 2010, the man noticed that the viral infections became more frequent and more severe.

The man started to take 300 mg/day doses of NBMI again in late 2011 and noticed that the infections, once again, got fewer and less severe. The man gradually decreased the dose of NBMI and halted the treatment by summer 2012.

Since then, the man sometimes takes NBMI when inflicted with a virus infection and has the impression that the infection is then less severe and shorter in duration than when not taking NBMI.

### Example 4

### Treatment of Patient with ME/CFS II

A man residing in the USA and diagnosed with myalgic encephalomyelitis/chronic fatigue syndrome (ME/CFS) had a long history of viral infections. The man was often sick and had, therefore, a poor quality of life.

The man reported that after starting to take 300 mg/day doses of NBMI in 2010, he no longer suffered from viral infections.

1. N,N-bis-2-mercaptoethyl isophthalamide, or a pharmaceutically-acceptable salt and/or derivative thereof, for use in a method of treatment or prevention of a viral infection.

2. The use of N,N-bis-2-mercaptoethyl isophthalamide, or a pharmaceutically-acceptable salt and/or derivative thereof, for the manufacture of a medicament for a method of treatment or prevention of a viral infection.

3. A method of treatment or prevention of a viral infection in a subject, which method comprises the administration of an effective amount of N,N-bis-2-mercaptoethyl isophthalamide, or a pharmaceutically-acceptable salt and/or derivative thereof, to a patient in need thereof.

4. A compound for use as claimed in claim 1, a use as claimed in claim 2, or a method as claimed in claim 3, wherein the viral infection is a respiratory tract infection.

5. A compound for use as claimed in claim 1, a use as claimed in claim 2, or a method as claimed in claim 3, wherein the respiratory tract infection, is an upper respiratory tract infection.

6. A compound for use, a use, or a method as claimed in claim 4 or claim 5, wherein the respiratory tract infection is caused by a virus selected from the group consisting of an influenza virus, a respiratory syncytial virus, a parainfluenza virus, a coronavirus, an adenovirus, a rhinovirus, or an enterovirus.

7. A compound for use as claimed in any one of claims 1 or 4 to 6, a use as claimed in any one of claims 2 or 4 to 6, or a method as claimed in any one of claims 3 to 6, wherein the treatment or prevention of the viral infection includes treatment or prevention of a disease that is being, or has been, caused by the virus.

8. A compound for use, a use, or a method as claimed in claim 7, wherein the virus is a respiratory virus.

9. A compound for use, a use, or a method as claimed in claim 8, wherein the disease is selected from common colds, influenza, sinus infections, tonsillitis, laryngitis, bronchitis, pneumonia, severe acute, respiratory syndrome, and bronchiolitis

10. A compound for use as claimed in any one of claims 1 or 4 to 9, a use as claimed in any one of claims 2 or 4 to 9, or a method as claimed in any one of claims 3 to 9, wherein the treatment includes treatment of the symptoms of a disease that is being, or has been, caused by the viral infection.

11. A compound for use, a use, or a method as claimed in claim 10, wherein the symptoms of the disease include one or more of cough, sneezing, nasal discharge, nasal congestion, fever, sore throat, headache, facial pressure, fatigue or weakness, myalgias, malaise, dyspnea, respiratory distress, respiratory failure and/or conjunctivitis.

12. A compound for use as claimed in any one of claims 1 or 4 to 11, a use as claimed in any one of claims 2 or 4 to 11, or a method as claimed in any one of claims 3 to 11, wherein the treatment includes prevention of virus-induced morbidity and/or mortality.

13. A pharmaceutical formulation for use in a method of treatment or of prevention of a viral respiratory tract infection comprising:
(a) N,N-bis-2-mercaptoethyl isophthalamide, or a pharmaceutically-acceptable salt and/or derivative thereof; and
(b) one or more other therapeutic agent that is useful in the treatment or prevention of viral infections,
and optionally one or more pharmaceutically-acceptable excipients.

14. A kit-of-parts for use in a method of treatment or of prevention of a viral respiratory tract infection comprising components:
(A) a pharmaceutical formulation comprising N,N-bis-2-mercaptoethyl isophthalamide, or a pharmaceutically-acceptable salt and/or derivative thereof, and optionally one or more pharmaceutically-acceptable excipients; and
(B) a pharmaceutical formulation comprising one or more other therapeutic agent that is useful in the treatment or prevention of viral infections, and optionally one or more pharmaceutically-acceptable excipients,
which components (A) and (B) are each provided in a form that is suitable for administration in conjunction with the other.
